SEQUENCE LISTING (29 sequences)

SEQ ID NO: 1 - protein, 7 aa, Artificial Sequence
  Synthetic
GSGGGGS

SEQ ID NO: 2 - protein, 17 aa, Artificial Sequence
  Synthetic
GSGGGGSGGGGSGGGGS

SEQ ID NO: 3 - protein, 12 aa, Artificial Sequence
  Synthetic
AEAAAKEAAAKA

SEQ ID NO: 4 - protein, 46 aa, Artificial Sequence
  Synthetic
AEAAAKEAAAKEAAAKEAAAKALEAEAAAKEAAAKEAAAKEAAAKA

SEQ ID NO: 5 - protein, 32 aa, Artificial Sequence
  Synthetic
AEAAAKEAAAKEAAAKEAAAKEAAAKEAAAKA

SEQ ID NO: 6 - protein, 7 aa, Artificial Sequence
  Synthetic
AEAAAKA

SEQ ID NO: 7 - protein, 17 aa, Artificial Sequence
  Synthetic
AEAAAKEAAAKEAAAKA

SEQ ID NO: 8 - protein, 22 aa, Artificial Sequence
  Synthetic
AEAAAKEAAAKEAAAKEAAAKA

SEQ ID NO: 9 - protein, 27 aa, Artificial Sequence
  Synthetic
AEAAAKEAAAKEAAAKEAAAKEAAAKA

SEQ ID NO: 10 - protein, 10 aa, Artificial Sequence
  Synthetic
APAPAPAPAP

SEQ ID NO: 11 - protein, 20 aa, Artificial Sequence
  Synthetic
APAPAPAPAPAPAPAPAPAP

SEQ ID NO: 12 - protein, 30 aa, Artificial Sequence
  Synthetic
APAPAPAPAPAPAPAPAPAPAPAPAPAPAP

SEQ ID NO: 13 - protein, 40 aa, Artificial Sequence
  Synthetic
APAPAPAPAPAPAPAPAPAPAPAPAPAPAPAPAPAPAPAP

SEQ ID NO: 14 - protein, 20 aa, Artificial Sequence
  Synthetic
PPPPPPPPPPPPPPPPPPPP

SEQ ID NO: 15 - protein, 30 aa, Artificial Sequence
  Synthetic
PPPPPPPPPPPPPPPPPPPPPPPPPPPPPP

SEQ ID NO: 16 - protein, 20 aa, Artificial Sequence
  Synthetic
SSSSSSSSSSSSSSSSSSSS

SEQ ID NO: 17 - protein, 40 aa, Artificial Sequence
  Synthetic
SSSSSSSSSSSSSSSSSSSSSSSSSSSSSSSSSSSSSSSS

SEQ ID NO: 18 - protein, 159 aa, Artificial Sequence
  Synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 19 - protein, 12 aa, Artificial Sequence
  Synthetic
GSGSGSGSGSGS

SEQ ID NO: 20 - protein, 11 aa, Artificial Sequence
  Synthetic
VSGWRLFKKIS

SEQ ID NO: 21 - protein, 13 aa, Artificial Sequence
  Synthetic
VSGWRLFKKISGS

SEQ ID NO: 22 - protein, 15 aa, Artificial Sequence
  Synthetic
GSVSGWRLFKKISGS

SEQ ID NO: 23 - protein, 184 aa, Artificial Sequence
  Synthetic
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGSGSGSGSGSGSVSGWRLFKKISGS

SEQ ID NO: 24 - DNA, 477 bp, Artificial Sequence
  Synthetic
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagt

SEQ ID NO: 25 - DNA, 33 bp, Artificial Sequence
  Synthetic
gtgagtggctggcgactgttcaagaagatcagc

SEQ ID NO: 26 - DNA, 39 bp, Artificial Sequence
  Synthetic
gtgagtggctggcgactgttcaagaagatcagcggcagc

SEQ ID NO: 27 - DNA, 45 bp, Artificial Sequence
  Synthetic
ggatccgtgagtggctggcgactgttcaagaagatcagcggcagc

SEQ ID NO: 28 - DNA, 555 bp, Artificial Sequence
  Synthetic
atggtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagtggtagcggtagcggtagcggtagcggtagcggatccgtgagtggctggcgactgttcaagaagatcagcggcagctaa

SEQ ID NO: 29 - DNA, 36 bp, Artificial Sequence
  Synthetic
ggtagcggtagcggtagcggtagcggtagcggatcc

(19) United States
(12) Patent Application Publication
(10) Pub. No.: US 2023/0257756 A1
(43) Pub. Date: Aug. 17, 2023

(54) REAL-TIME CELLULAR THERMAL SHIFT ASSAY (RT-CETSA) FOR RESEARCH AND DRUG DISCOVERY

(71) Applicant: The United States of America,as represented by the Secretary,Department of Health and Human Services, Bethesda, MD (US)

(72) Inventors: Mark J. Henderson, Damascus, MD (US); Michael H. Ronzetti, Silver Spring, MD (US); Bolormaa Baljinnyam, Rockville, MD (US); Tino W. Sanchez, Rockville, MD (US); Samuel G. Michael, Germantown, MD (US); Ashley E. Owens, Murfreesboro, TN (US); Anton Simeonov, Bethesda, MD (US)

(73) Assignee: The United States of America,as represented by the Secretary,Department of Health and Human Services, Bethesda, MD (US)

(21) Appl. No.: 18/020,843

(22) PCT Filed: Aug. 9, 2021
(86) PCT No.: PCT/US2021/045184

§ 371 Date: Feb. 10, 2023

Related U.S. Application Data
(60) Provisional application No. 63/063,689, filed on Aug. 10, 2020.

(51) Int. Cl. C12N 15/62; C12N 9/06; C07K 14/705; C12N 15/86; G01N 33/68

(52) U.S. Cl. CPC C12N 15/625; C12Y 101/01027; C12Y 105/01003; C12N 9/003; C07K 14/70596; C07K 14/70521; C12N 15/86; G01N 33/6845; C12N 2330/51

(57) ABSTRACT

The disclosure provides methods for carrying out Real Time Cellular Thermal Shift Assays (RT-CETSA). Also provided are molecular constructs and protein constructs for use in such assays and devices suitable for carrying out such assays.

### CROSS-REFERENCE TO RELATED APPLICATION

This patent application claims the benefit of co-pending U.S. Provisional Patent Application No. 63/063,689 filed Aug. 10, 2020, which is incorporated by reference in its entirety herein.

### STATEMENT REGARDING FEDERALLY SPONSORED RESEARCH OR DEVELOPMENT

This invention was made with Government support by the National Institutes of Health, National Center for Advancing Translational Sciences. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 11,893 byte ASCII (Text) file named “754641_ST25.txt,” created on Aug. 6, 2021.

### BACKGROUND

The Cellular Thermal Shift Assay (CETSA) is an experimental procedure that enables, e.g., the assessment of drug-protein interaction by quantifying changes in the thermal stability of a protein upon ligand binding. However, the original CETSA protocol is significantly low-throughput, requires substantial optimization, and relies on time-consuming western blot detection of the target of interest. Further, traditional CETSA is limited to either a single temperature or a single compound concentration across a temperature range. In a modified CETSA method, the protein of interest is tagged with nanoLuciferase, a commonly-used luminescent protein, and, subsequently, the luciferase substrate is added to measure levels of the protein of interest. Then, the intact thermally-stable target of interest can be quantified by a luminescent signal. However, nanoLuciferase melts at too low a temperature to be used in a full CETSA temperature ramp.

Accordingly, new methods and reporter molecules are needed which allow researchers to view thermal shift data via a luminescent signal, in real time, and across a full CETSA temperature ramp in order to generate, e.g., full aggregation profiles for multiple samples in parallel.

### BRIEF SUMMARY

The disclosure provides protein constructs comprising, consisting of, or consisting essentially of a target protein of interest, a first peptide linker, and a reporter region, wherein the reporter region comprises, consists of, or consists essentially of, from N-Terminus to C-Terminus, a LgBiT fragment, a second peptide linker, and a HiBiT fragment.

Also provided are biological vectors encoding the protein constructs.

Also provided is a method for utilizing the protein constructs to test samples, wherein the samples comprise, consist of, or consist essentially of living intact cells. The method comprises, consists of, or consists essentially of:
a) transfecting the cells with a biological vector encoding the protein construct under conditions suitable to allow the expression of the protein construct within the cells;
b) exposing the protein construct to a photon generating substrate (e.g. furimazine); and
c) exposing the cells to an increasing temperature gradient while detecting the change in luminescence of the sample in real time.

Also provided is an analytical device; wherein the analytical device is capable of simultaneously heating and collecting real time luminescence data for multiple samples; the device comprising: (a) a thermal cycler block adapted to receive a multi-well plate comprising, consisting of, or consisting essentially of the multiple samples, and (b) a detection device capable of detecting luminescence, wherein the camera is positioned such that it can detect changing luminescence in the multiple samples in real time over a range of temperatures.

### BRIEF DESCRIPTION OF THE SEVERAL VIEWS OF THE DRAWING(S)

FIG. 1A is a graph depicting the melting points for three different proteins by differential scanning fluorimetry. As can be seen in the graph, the LgBiT when incubated with a peptide comprising, consisting of, or consisting essentially of the HiBit fragment of NanoLuc with GlySer extensions had an increased melting point relative to the LgBiT fragment by itself and 156+Native peptide.

FIG. 1B provides a visual depiction of an aspect of the reporter region of the disclosed protein constructs with ThermLuc as carboxy-terminal fusion (top) or amino-terminal fusion (bottom). The locations of Linker 1 and linker 2 are depicted.

FIG. 1C depicts a comparison of the melting profile of 11s-86b fusion proteins with varying Gly-Ser linker lengths when expressed in HEK293T cells. When the size of the linker was increased to 3 or more GlySer repeats between the two fragments, the resultant fusion protein displayed minimal melting over the temperature ramp range commonly used in CETSA experiments.

FIG. 1D depicts the amino acid sequence (SEQ ID NO: 23) of the reporter region designated “ThermLuc”.

FIG. 1E depicts a nucleotide sequence (SEQ ID NO: 28) encoding the reporter region designated “ThermLuc”.

FIG. 1F depicts a comparison of the melting profile of 11s-86b fusion proteins with varying gly-ser linker lengths when expressed in HEK293T cells and analyzed using the real-time analytical device according to the present disclosure. When the size of the linker was increased to 3 or greater GlySer repeats between the two fragments, the resultant fusion protein displayed less melting than Nanoluciferase over the temperature ramp range commonly used in CETSA experiments.

FIG. 1G depicts the melting profile of 11s-86b fusion proteins with varying gly-ser linker lengths when expressed in HEK293T cells and analyzed using the real-time analytical device Luminescence values are normalized to the 37° C. value for each fusion protein.

FIG. 2 visually depicts the configuration of an analytical device according to the present disclosure.

FIG. 3A depicts a still image of a plate containing LDHA-ThermLuc transfected HEK293T cells during an RT-CETSA assay. This still image is representative of a single time point in the continuous, real-time, visualization of luminescence provided by RT-CETSA.

FIG. 3B depicts the results of an experiment utilizing HEK293T cells wherein the target protein of interest is LDHA fused to either ThermLuc or Nanoluciferase, where thermal shift is only detectable for the ThermLuc fusion.

FIG. 3C depicts results indicating that some compounds may show binding in the DSF assay with purified protein, but not CETSA, because they do not bind the target in cells, for instance because they lack membrane permeability.

FIG. 3D depicts data regarding thermal shifts across a multi-well plate.

FIG. 3E depicts data regarding thermal shifts across a multi-well plate.

FIG. 3F depicts data regarding thermal shifts across a multi-well plate.

FIG. 3G presents a visual depiction of data indicating that LDHA inhibitors show a dose dependent shift.

FIG. 3H depicts the RT-CETSA melt profile of eight examples of ThermLuc fused to target proteins of interest.

FIG. 3I depicts target engagement in RT-CETSA.

FIG. 3J depicts target engagement in RT-CETSA.

FIG. 3K depicts target engagement in RT-CETSA.

FIG. 3L is a visual depiction of EC50 values for twenty two LDHA inhibitors (calculated based on area under the curve or T_{agg} metrics) calculated for LDHA-ThermLuc using RT-CETSA as compared to SplitLuc CETSA.

FIG. 4A provides a graph indicating that the melting of target proteins (exemplified by LDHA-ThermLuc) occurs within seconds of the application of heat. Accordingly, RT-CETSA allows for a real-time read out of target melting and small molecule engagement with temporal resolution that cannot be obtained with other CETSA techniques.

FIG. 4B depicts the thermal aggregation profile of immunotherapeutic targets obtained with traditional CETSA (as compared to RT-CETSA in FIG. 4C).

FIG. 4C depicts the thermal aggregation profile of immunotherapeutic targets obtained with RT-CETSA (as compared to traditional CETSA in FIG. 4B).

FIG. 5 depicts an example of a multi-target readout obtained by RT-CETSA.

FIG. 6 provides a visual depiction of ThermLuc being inserted into a pcDNA3.1 vector with proper In-Fusion (Takara Bio) homologous sequences, containing a BamHI restriction site (encoding Gly-Ser) at the junction between ThermLuc and the target.

FIG. 7A is a schematic diagram illustrating dihydrofolate reductase (DHFR)-ThermLuc constructs that were prepared with various first peptide linker region sequences between the target of interest (i.e. DHFR) and the reporter molecule (i.e. ThermLuc) (SEQ ID NOS: 1-17).

FIG. 7B is a graph depicting the cellular thermal melt profiles of DHFR fusion proteins comprising NanoLuc and ThermLuc.

FIG. 7C is a graph depicting the thermal stability of DHFR-ThermLuc fusions when separated by various first peptide linker sequences.

### DETAILED DESCRIPTION

### Protein Constructs

The disclosure provides protein constructs comprising, consisting of, or consisting essentially of a target protein of interest, a first peptide linker, and a reporter region.

In aspects, the protein construct comprises, consists of, or consists essentially of, from N-terminus to C-terminus, the target protein of interest, the first peptide linker, and the reporter region. In other aspects, the protein construct comprises, consists of, or consists essentially of, from N-terminus to C-terminus, the reporter region, the first peptide linker, and the target protein of interest.

The target protein of interest may be any protein. In some aspects the target protein of interest may be a full length protein. The target protein of interest may be, for example any protein in the proteome of a mammalian cell. The target protein of interest may also be a protein fragment. The target protein of interest may also be a complex of multiple peptides or proteins. Examples of suitable target proteins of interest include potential pharmaceutical targets, proteins involved in the inflammatory process, proteins involved in regulating the cell cycle, proteins involved in cancer cell proliferation or cancer cell metabolism, proteins involved in disease or other pathologies, and proteins with unknown functions.

Examples of specific target proteins of interest include, without limitation, lactate dehydrogenase A (“LDHA”), mammalian tyrosine-protein kinase ABL1 (“c-Abl”), B-lymphocyte antigen CD19 (“CD19”), B-lymphocyte antigen CD20 (“CD20”), programmed cell death protein 1 (“PD1”), cytotoxic T-lymphocyte-associated protein 4 (“CTLA4”), nerve growth factor (“NGF”), dihydrofolate reductase (“DHFR”) and proprotein convertase subtilisin/kexin type 9 (“PCSK9”). In aspects, the target protein of interest may be a protein that has been modified relative to wild-type. Examples of modified proteins include, for example, proteins in which one or more point mutations have been introduced.

In aspects, the target protein of interest is LDHA, DHFR, cAbl, CD19, CD20, PD1, CTLA 4, NGF or PCSK9.

The first peptide linker may be any suitable sequence of amino acids. In aspects, the sequence of the first peptide linker comprises, consists of, or consists essentially of a polypeptide having greater than 80% identity with any one of SEQ ID NOS: 1-17. In aspects, the sequence of the first peptide linker comprises, consists of, or consists essentially of a polypeptide having greater than 85% identity with any one of SEQ ID NOS: 1-17. In aspects, the sequence of the first peptide linker comprises, consists of, or consists essentially of a polypeptide having greater than 90% identity with an one of SEQ ID NOS: 1-17. In aspects, the sequence of the first peptide linker comprises, consists of, or consists essentially of a polypeptide having greater than 95% identity with any one of SEQ ID NOS: 1-17. In aspects, the sequence of the first peptide linker comprises, consists of, or consists essentially of any one of SEQ ID NOS: 1-17.

The reporter region comprises, consists of, or consists essentially of, from N-terminus to C-terminus, a LgBiT fragment, a second peptide linker, and a HiBiT fragment.

In aspects, the LgBiT fragment is a fragment of nanoluciferase (“NanoLuc”), which is also referred to herein as “11s”. 11s has the amino acid sequence of SEQ ID NO: 18. In other aspects, the LgBiT fragment comprises, consists of, or consists essentially of a polypeptide having at least 80% identity with SEQ ID NO: 18. In other aspects, the LgBiT fragment comprises, consists of, or consists essentially of a polypeptide having at least 85% identity with SEQ ID NO: 18. In other aspects, the LgBiT fragment comprises, consists of, or consists essentially of a polypeptide having at least 90% identity with SEQ ID NO: 18. In other aspects, the LgBiT fragment comprises, consists of, or consists essentially of a polypeptide having at least 95% identity with SEQ ID NO: 18.

The second peptide linker comprises, consists of, or consists essentially of any suitable sequence of amino acids. In some aspects, the second peptide linker comprises, consists of, or consists essentially of one or more glycine-serine (“GlySer”) repeats. In some aspects the second peptide linker comprises, consists of, or consists essentially of one GlySer repeat, two GlySer repeats, three GlySer repeats, four GlySer repeats, five GlySer repeats or six GlySer repeats. In other aspects the second peptide linker comprises, consists of, or consists essentially of greater than 6 GlySer repeats. In aspects, the second peptide linker comprises, consists of, or consists essentially of seven GlySer repeats, eight GlySer repeats, nine GlySer repeats, ten GlySer repeats, eleven GlySer repeats, twelve GlySer repeats, thirteen GlySer repeats, fourteen GlySer repeats or fifteen GlySer repeats. In one aspect, the second peptide linker is 6 GlySer repeats (SEQ ID NO: 19).

In aspects, the HiBiT fragment is a fragment of NanoLuc having the amino acid sequence of SEQ ID NO: 20. In other aspects, the HiBiT fragment comprises, consists of, or consists essentially of a polypeptide having at least 80% identity with SEQ ID NO: 20. In other aspects, the HiBiT fragment comprises, consists of, or consists essentially of a polypeptide having at least 85% identity with SEQ ID NO: 20. In other aspects, the HiBiT fragment comprises, consists of, or consists essentially of a polypeptide having at least 90% identity with SEQ ID NO: 20. In other aspects, the HiBiT fragment comprises, consists of, or consists essentially of a polypeptide having at least 95% identity with SEQ ID NO: 20.

In aspects, the HiBiT fragment additionally comprises, consists of, or consists essentially of one or more GlySer extensions. For example, in an aspect a GlySer extension is present at the C-terminus of the HiBit fragment. In one such aspect the HiBiT fragment comprises, consists of, or consists essentially of SEQ ID NO: 20 with GlySer at the C-terminus (SEQ ID NO: 21). In other aspects that HiBiT fragment comprises, consists of, or consists essentially of a polypeptide having at least 80% identity with SEQ ID NO: 21. In other aspects the HiBiT fragment comprises, consists of, or consists essentially of a polypeptide having at least 85% identity with SEQ ID NO: 21. In other aspects that HiBiT fragment comprises, consists of, or consists essentially of a polypeptide having at least 90% identity with SEQ ID NO: 21. In other aspects that HiBiT fragment comprises, consists of, or consists essentially of a polypeptide having at least 95% identity with SEQ ID NO: 21.

In other aspects, the HiBiT fragment comprises, consists of, or consists essentially of a GlySer extension at both the N-Terminus and the C-terminus. In one such aspect, the HiBiT fragment comprises, consists of, or consists essentially of SEQ ID NO: 20 with GlySer at the N-terminus and GlySer at the C-terminus. This peptide sequence is referred to as “86b” (SEQ ID NO: 22). In other aspects that HiBiT fragment comprises, consists of, or consists essentially of a polypeptide having at least 80% identity with SEQ ID NO: 22. In other aspects that HiBiT fragment comprises, consists of, or consists essentially of a polypeptide having at least 85% identity with SEQ ID NO: 22. In other aspects that HiBiT fragment comprises, consists of, or consists essentially of a polypeptide having at least 90% identity with SEQ ID NO: 22. In other aspects that HiBiT fragment comprises, consists of, or consists essentially of a polypeptide having at least 95% identity with SEQ ID NO: 22.

In aspects, the reporter region is a polypeptide having the amino acid sequence of SEQ ID NO: 23 (referred to as “ThermLuc”). In other aspects, the reporter region comprises, consists of, or consists essentially of a polypeptide having at least 80% identity with SEQ ID NO: 23. In other aspects, the reporter region comprises, consists of, or consists essentially of a polypeptide having at least 85% identity with SEQ ID NO: 23. In other aspects, the reporter region comprises, consists of, or consists essentially of a polypeptide having at least 90% identity with SEQ ID NO: 23. In other aspects, the reporter region comprises, consists of, or consists essentially of a polypeptide having at least 95% identity with SEQ ID NO: 23.

### Biological Vector

Aspects of the disclosure comprise a biological vector encoding the protein construct.

In an aspect of the disclosure, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises, consists of, or consists essentially of a nucleotide sequence encoding the mRNA, protein, polypeptide, or peptide, and the vector is contacted with the cell under conditions sufficient to have the mRNA, protein, polypeptide, or peptide expressed within the cell. The disclosed vectors are not naturally-occurring as a whole. However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vectors can be prepared using standard recombinant DNA techniques. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the disclosed expression vectors include, for instance, neomycin/G418 resistance genes, hygromycin resistance genes, histidinol resistance genes, tetracycline resistance genes, and ampicillin resistance genes.

The vector may further comprise regulatory sequences that are operably linked to the nucleotide sequence encoding the protein constructs which permits one or more of the transcription, translation, and expression protein constructs in a cell transfected with the vector or infected with a virus that comprises, consists of, or consists essentially of the vector. As used herein, “operably linked” sequences include both regulatory sequences that are contiguous with the nucleotide sequence encoding the protein construct and regulatory sequences that act in trans or at a distance to control the nucleotide sequence encoding the protein construct.

The regulatory sequences may include appropriate transcription initiation, termination, promoter and enhancer sequences; RNA processing signals such as splicing and polyadenylation (polyA) signal sequences; sequences that stabilize cytoplasmic mRNA; sequences that enhance translation efficiency (i.e., Kozak consensus sequence); sequences that enhance protein stability.

In aspects, the biological vector comprises, consists of, or consists essentially of a promotor that drives expression of the protein construct. The promoter may be any promoter suitable for expressing the protein construct in a target cell, e.g., a mammalian cell. The promoter may be inducible or constitutive. In an aspect of the disclosure, the promoter is suitable for expressing the protein construct in a particular cell type. In this regard, the promoter may be cell-specific.

In some aspects, the vector is a pcDNA3.1 vector.

In some aspects, the vector is a viral vector. Examples of suitable viral vectors include retroviral vectors, lentiviral vectors, adenoviral vectors, adeno-associated viral (AAV) vectors.

In some aspects, the biological vector is prepared by inserting the sequence encoding the protein construct into a universal acceptor plasmid. For example, a nucleotide sequence encoding the protein construct may be inserted into a pcDNA3.1 vector with proper In-Fusion consensus sequences.

In aspects, the biological vector comprises, consists of, or consists essentially of nucleotides encoding the LgBiT fragment of the protein construct, for example SEQ ID NO: 24. In other aspects, the biological vector comprises, consists of, or consists essentially of a nucleotide sequence having greater than 80% identity to SEQ ID NO: 24. In other aspects the biological vector comprises, consists of, or consists essentially of a nucleotide sequence having greater than 85% identity to SEQ ID NO: 24. In other aspects the biological vector comprises, consists of, or consists essentially of a nucleotide sequence having greater than 90% identity to SEQ ID NO: 24. In other aspects the biological vector comprises, consists of, or consists essentially of a nucleotide sequence having greater than 95% identity to SEQ ID NO: 24.

In aspects, the biological vector comprises, consists of, or consists essentially of nucleotides encoding the HiBiT fragment of the protein construct, for example, SEQ ID NOS: 25, 26 or 27. In aspects the biological vector comprises, consists of, or consists essentially of a nucleotide sequence having greater than 80% identity to SEQ ID NO: 25. In other aspects the biological vector comprises, consists of, or consists essentially of a nucleotide sequence having greater than 85% identity to SEQ ID NO: 25. In other aspects the biological vector comprises, consists of, or consists essentially of a nucleotide sequence having greater than 90% identity to SEQ ID NO: 25. In other aspects the biological vector comprises, consists of, or consists essentially of a nucleotide sequence having greater than 95% identity to SEQ ID NO: 25. In aspects, the biological vector comprises, consists of, or consists essentially of the nucleotide sequence SEQ ID NO: 26. In other aspects the biological vector comprises, consists of, or consists essentially of a nucleotide sequence having greater than 80% identity to SEQ ID NO: 26. In other aspects the biological vector comprises, consists of, or consists essentially of a nucleotide sequence having greater than 85% identity to SEQ ID NO: 26. In other aspects the biological vector comprises, consists of, or consists essentially of a nucleotide sequence having greater than 90% identity to SEQ ID NO: 26. In other aspects the biological vector comprises, consists of, or consists essentially of a nucleotide sequence having greater than 95% identity to SEQ ID NO: 26. In aspects, the biological vector comprises, consists of, or consists essentially of the nucleotide sequence SEQ ID NO: 27. In other aspects the biological vector comprises, consists of, or consists essentially of a nucleotide sequence having greater than 80% identity to SEQ ID NO: 27. In other aspects the biological vector comprises, consists of, or consists essentially of a nucleotide sequence having greater than 85% identity to SEQ ID NO: 27. In other aspects the biological vector comprises, consists of, or consists essentially of a nucleotide sequence having greater than 90% identity to SEQ ID NO: 27. In other aspects the biological vector comprises, consists of, or consists essentially of a nucleotide sequence having greater than 95% identity to SEQ ID NO: 27.

In aspects, the biological vector comprises, consists of, or consists essentially of a nucleotide sequence encoding ThermLuc, for example SEQ ID NO: 28. In aspects the biological vector comprises, consists of, or consists essentially of a nucleotide sequence having greater than 80% identity to SEQ ID NO: 28. In other aspects the biological vector comprises, consists of, or consists essentially of a nucleotide sequence having greater than 85% identity to SEQ ID NO: 28. In other aspects the biological vector comprises, consists of, or consists essentially of a nucleotide sequence having greater than 90% identity to SEQ ID NO: 28. In other aspects the biological vector comprises, consists of, or consists essentially of a nucleotide sequence having greater than 95% identity to SEQ ID NO: 28.

In aspects, the biological vector comprises, consists of, or consists essentially of a nucleotide sequence encoding a second peptide linker. In aspects, the nucleotide sequence encodes a second peptide linker comprising Gly-Ser repeats, for example six Gly-Ser repeats. In aspects, the biological vector comprises, consists of, or consists essentially of a nucleotide sequence comprising SEQ ID NO: 29.

### RT-CETSA Method

Another aspect of the disclosure is a method for utilizing the disclosed protein constructs to test samples, wherein the samples comprise living, intact cells. The method comprises, consists of, or consists essentially of:
a) transfecting the cells with a biological vector encoding the protein construct under conditions suitable to allow the expression of the protein construct within the cells;
c) exposing the expressed protein construct to a photon generating substrate; and
d) exposing the cells to an increasing temperature gradient while detecting the change in luminescence of the sample in real time.

In aspects, the method is a Real Time Cellular Thermal Shift Assay, which allows researchers to view thermal shift data in real time.

The cells for use in the disclosed methods may be any suitable cells. For example, the cells may be mammalian cells. In some aspects, the cells are human. Examples of suitable mammalian cell lines include the Chinese hamster ovary (CHO), COS, and human cell lines such as HEK and HeLa. In some aspects, the cells are HEK293T cells. The cells may be cultured in any suitable media according to methods known in the art. For example HEK293T cells may be cultured in DMEM, 4.5 g/L glucose (Invitrogen) with 10% fetal bovine serum (FBS), 6 mM L-glutamine, 1 mM sodium pyruvate, 50 U/mL penicillin, and 50 μg/mL streptomycin. In an aspect, the cells may be in placed into suspension. In aspects, the suspension comprising, consisting of, or consisting essentially of the cells may be transferred to wells in multi-well plates.

In aspects, the biological vector is transfected into the cells. A number of transfection techniques are generally known in the art (see, e.g., Graham et al., Virology, 52: 456-467 (1973); Sambrook et al., supra; Davis et al., Basic Methods in Molecular Biology, Elsevier (1986); and Chu et al., Gene, 13: 97 (1981). Transfection methods include calcium phosphate co-precipitation (see, e.g., Graham et al., supra), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Felgner et al., Proc. Natl. Acad. Sci. USA, 84: 7413-7417 (1987)), and nucleic acid delivery using high velocity microprojectiles (see, e.g., Klein et al., Nature, 327: 70-73 (1987)). In some aspects, the vector is transiently transfected into the cells. In other aspects, stable transfection is utilized. Transfection, as used herein, also refers to viral transduction in aspects wherein the biological vector is a viral vector.

In an aspect, the expressed protein construct is contacted with a photon generating substrate. An example of a photon generating substrate is furimazine. This can be achieved via any appropriate laboratory technique. For example, where the cells are in suspension, a suitable amount of furimazine may be added to the suspension.

Aspects of the disclosure comprise exposing the cells to an increasing temperature gradient. “Increasing temperature gradient”, as used herein, refers to a temperature that increases from a starting temperature to a final temperature over time. Different starting temperatures may be used, for example about 20° C., about 30° C., about 40° C. In many aspects the starting temperature will be between 30° C. and 37° C. In some aspects, the starting temperature will be room temperature. Different final temperatures may also be used, for example about 90° C., 80° C., 70° C., 60° C., or 50° C. Any suitable combination of starting and final temperatures may be used. The increasing temperature gradient may have a starting temperature of, for example, 20° C. and an ending temperature of about 80° C. Alternatively, the increasing temperature gradient may have a have a starting temperature of about 30° C. and an ending temperature of about 70° C. As another example, the increasing temperature gradient may have a starting temperature of about 40° C. and an ending temperature of about 60° C. Any suitable rate of temperature increase may be used in the increasing temperature gradient. In one aspect the rate is about 0.2° C. per second. Alternatively, faster or slower rates of increase may be used.

Aspects of the method further comprise contacting the protein construct with one or more additional test molecules. In such aspects, the RT-CETSA assay may be used to detect binding (or lack thereof) between the target protein of interest and the test molecule in the cells. The additional test molecule may be a small molecule. In aspects, the additional test molecule binds to the target protein of interest. Examples of suitable small molecules include, without limitation, potential drug candidates, ligands known to bind to the target protein(s) of interest, known inhibitors of the target protein(s) of interest, and molecules with unknown biological activity.

The additional test molecule(s) may also be larger molecules such as, for example, proteins and antibodies. In some aspects, the additional test molecule is a monoclonal antibody. Methods for obtaining and preparing monoclonal antibodies are known to those skilled in the art.

Aspects of the disclosure also allow for the parallel testing of multiple samples with different temperatures of aggregation in a high-throughput environment. The samples may contain different target proteins of interest and/or different additional test molecules from one another. Such samples may be assayed together in, for example, a multi-well plate.

### Analytical Device

An aspect of the disclosure is an analytical device; wherein the analytical device is capable of simultaneously collecting real time luminescence data during a temperature hold or ramp for multiple samples. In an aspect, the device comprises, consists of, or consists essentially of: (a) a thermal cycler block adapted to receive a multi-well plate comprising, consisting of, or consisting essentially of the multiple samples; (b) a detection device capable of detecting luminescence; and (c) a thermal top-heat assembly adapted to maintain even heating across the top of the multi well plate and to allow a luminescent signal to pass through to the detection device. The detection device is positioned such that it can detect changing luminescence in the multiple samples in real time.

In aspects, the analytical device comprises, consists of, or consists essentially of a thermal cycler. A thermal cycler, as disclosed herein, is a laboratory apparatus typically used to amplify segments of DNA via the polymerase chain reaction. A suitable thermal cycler is capable of applying heat to the samples being tested to achieve an increasing temperature gradient consistent across the multiwall plate. Thermal cyclers according to the present disclosure comprise, consist of, or consist essentially of a thermal block adapted to receive samples. In an aspect, the block is adapted to receive one or more multi-well sample plates. Aspects of the disclosure include the modification of commercially available thermal cyclers by, e.g., removing excitation and emissions filters and/or exchanging detection devices to increase sensitivity to luminescence.

Suitable detection devices for detecting luminescence are known to those skilled in the art. For example, in an aspect the detection device is a sensitive CCD or CMOS sensor. The sensor may be cooled, for example, water cooled. An example of a suitable CCD camera is the ORCA II (Hamamatsu).

In aspects, the thermal cycler is adapted to receive multi well plates. Such plates include 96-well plates, 384-well plates, and 1536-well plates, all of which are readily available and familiar to those skilled in the art.

Aspects also include a thermal top-heat assembly that is positioned above the multi-well plate and is adapted to ensure event heating across the plate without impeding the luminescent signal.

### Aspects of the Disclosure

1. A protein construct comprising a target protein of interest, a first peptide linker, and a reporter region, wherein the reporter region comprises, from N-terminus to C-terminus, an LgBiT fragment, a second peptide linker, and an HiBiT fragment.

2. The protein construct of aspect 1, wherein the protein construct comprises from N-terminus to C-terminus the target protein of interest, the first peptide linker, and the reporter region.

3. The protein construct of aspect 1, wherein the protein construct comprises from N-terminus to C-terminus, the reporter region, the first peptide linker, and the target protein of interest.

4. The protein construct of any one of aspects 1-3 wherein the target protein of interest is LDHA, DHFR, cAbl, CD19, CD20, PD1, CTLA 4, NGF or PCSK9.

5. The protein construct of any one of aspects 1-4, wherein the first peptide linker comprises a polypeptide having at least 80% identity with any one of SEQ ID NOS: 1-17.

6. The protein construct of any one of aspects 1-5, wherein the LgBiT fragment comprises a polypeptide having at least 80% identity with SEQ ID NO: 18.

7. The protein construct of any one of aspects 1-6, wherein the second peptide linker comprises one or more GlySer repeats.

8. The protein construct of aspect 7, wherein the second peptide linker comprises SEQ ID NO: 19.

9. The protein construct of any one of aspects 1-8, wherein the HiBiT fragment comprises a polypeptide having at least 80% identity with SEQ ID NO: 20.

10. The protein construct of aspect 9 wherein the HiBiT fragment additionally comprises one or more GlySer extensions.

11. The protein construct of aspect 10 wherein the HiBiT fragment comprises a polypeptide having at least 80% identity with SEQ ID NO: 21.

12. The protein construct of aspect 10 wherein the HiBiT fragment comprises a polypeptide having at least 80% identity with SEQ ID NO: 22.

13. The protein construct of any one of aspects 1-12, wherein the reporter region comprises a polypeptide having at least 80% identity with SEQ ID NO: 23.

14. A biological vector encoding a protein construct according to any one of aspects 1-13.

15. The biological vector of aspect 14 wherein the vector is a recombinant expression vector comprising a promoter that drives expression of the of the protein construct in mammalian cells.

16. The biological vector of aspect 14 or 15, wherein the vector is a universal acceptor plasmid.

17. The biological vector of any one of aspects 14-16, wherein the vector is a pcDNA3.1 vector.

18. The biological vector of aspect 14 wherein the vector is a viral vector.

19. The biological vector of any one of aspects 14-18 wherein the biological vector comprises a nucleotide sequence having greater than 80% identity to SEQ ID NO: 24.

20. The biological vector of aspects 14-19 wherein the biological vector comprises a nucleotide sequence having greater than 80% identity to SEQ ID NO: 25, 26, or 27.

21. The biological vector of aspect 14-20 wherein the biological vector comprises a nucleotide sequence having greater than 80% identity to SEQ ID NO: 26.

22. The biological vector of aspects 14-18 wherein the biological vector comprises a nucleotide sequence having greater than 80% identity to SEQ ID NO: 28.

23. A method for testing one or more samples, wherein the one or more samples comprise living intact cells, the method comprising:
a) transfecting the cells with the biological vector of any one of aspects 14-22 under conditions suitable to allow the expression of the protein construct within the cells;
c) exposing the expressed protein construct to a photon generating substrate; and
d) exposing the cells to an increasing temperature gradient while detecting the change in luminescence of the sample in real time.

24. The method of aspect 23, wherein the photon generating substrate is furimazine.

25. The method of aspect 23 or aspect 24, further comprising contacting the protein construct with one or more additional test molecules prior to exposing the cells to the increasing temperature gradient.

26. The method of aspect 25 wherein the additional test molecule is a small molecule.

27. The method of aspect 25 or 26 wherein the additional test molecule binds to the target protein of interest.

28. The method of any one of aspects 25-27 wherein the additional test molecule is a binder (e.g. inhibitor) of the target protein of interest.

29. The method of aspect 25 wherein the additional test molecule is an antibody.

30. The method of any one of aspects 23-29, wherein two or more samples are tested in parallel.

31. The method of aspect 30, wherein the two or more samples comprise different protein constructs.

32. The method of any one of aspects 30-31, wherein the two or more samples comprise different target proteins.

33. The method of any one of aspects 30-32 wherein the protein constructs of each of the two or more samples is contacted with one or more additional test molecules prior to exposing the cells to the increasing temperature gradient.

34. An analytical device; wherein the analytical device is capable of simultaneously heating and collecting real time luminescence data for multiple samples; the device comprising:
(a) a thermal cycler block adapted to receive a multi-well plate comprising the multiple samples;
(c) a detection device capable of detecting luminesce; and
(b) a thermal top-heat assembly adapted to maintain even heating across the top of the multi-well plate and to allow a luminescent signal to pass through to the detection device; wherein the detection device is positioned such that it can detect changing luminescence in the multiple samples in real time over a range of temperature.

35. The analytical device of aspect 34 wherein the detection device is a CCD sensor or a CMOS sensor.

36. The analytical device of aspect 34 or 35, wherein the multi well plate is a 96-well plate, a 384-well plate or a 1,536 well plate.

It shall be noted that the preceding are merely examples of aspects. Other exemplary aspects are apparent from the entirety of the description herein. It will also be understood by one of ordinary skill in the art that each of these aspects may be used in various combinations with the other aspects provided herein.

### EXAMPLES

The following examples should not be construed as in any way limiting the scope of the present disclosure.

### Example 1

This example describes the development of a thermally stable nanoLuciferase-based reporter molecule for use in Real Time CETSA experiments.

The nanoLuciferase (NanoLuc) enzyme is a commonly used and highly-luminescent 19.1 kDa reporter molecule. However, its lower melting temperature (58° C.) would mask most ligand-induced stabilization and falsely shift the apparent temperature of aggregation, or T_{agg}, because the melting of NanoLuc would drive aggregation rather than the melting of the protein of interest.

To develop a thermally stable NanoLuc based reporter molecule, the inventors investigated the characteristics of fragments of NanoLuc. 11S refers to the LgBit fragment of NanoLuc, the amino acid sequence of which is provided as SEQ ID NO: 18.

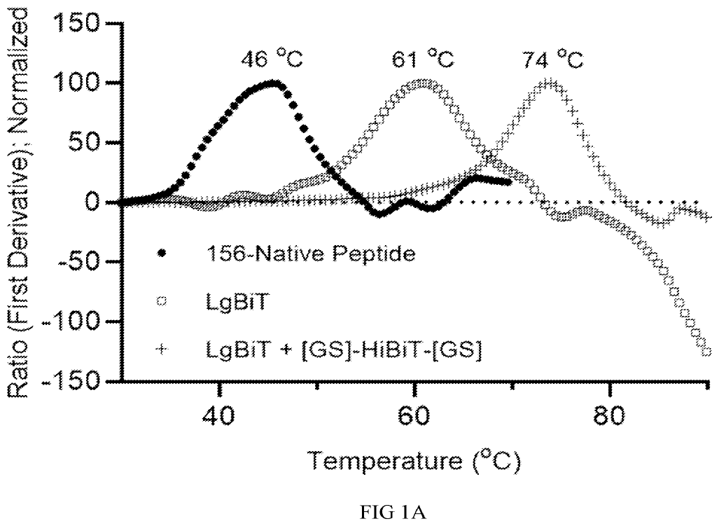

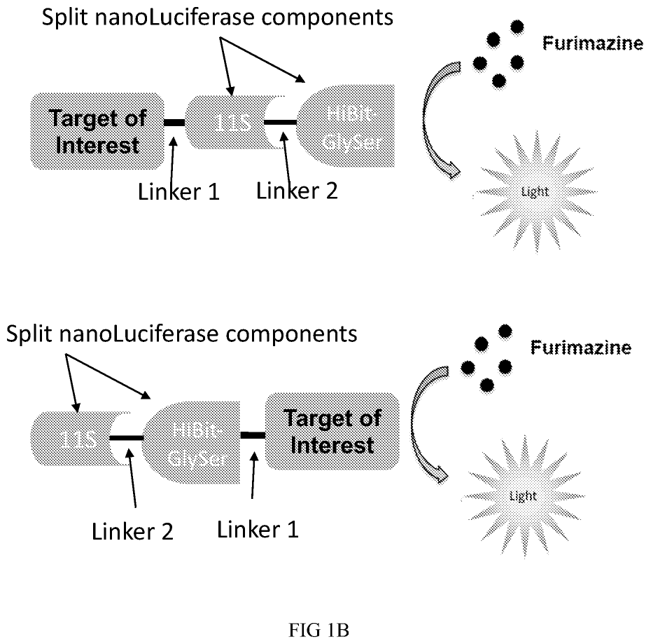

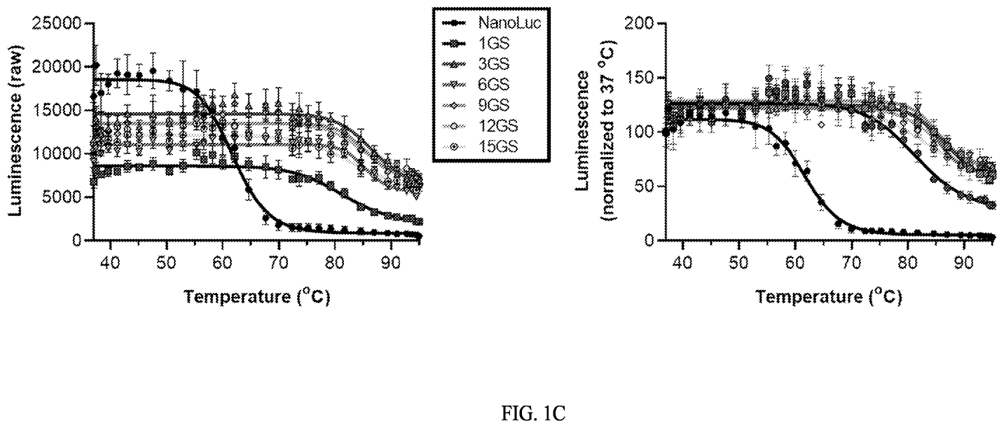

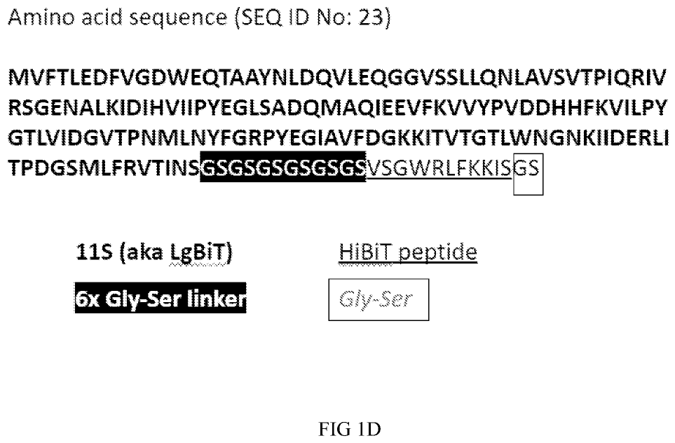

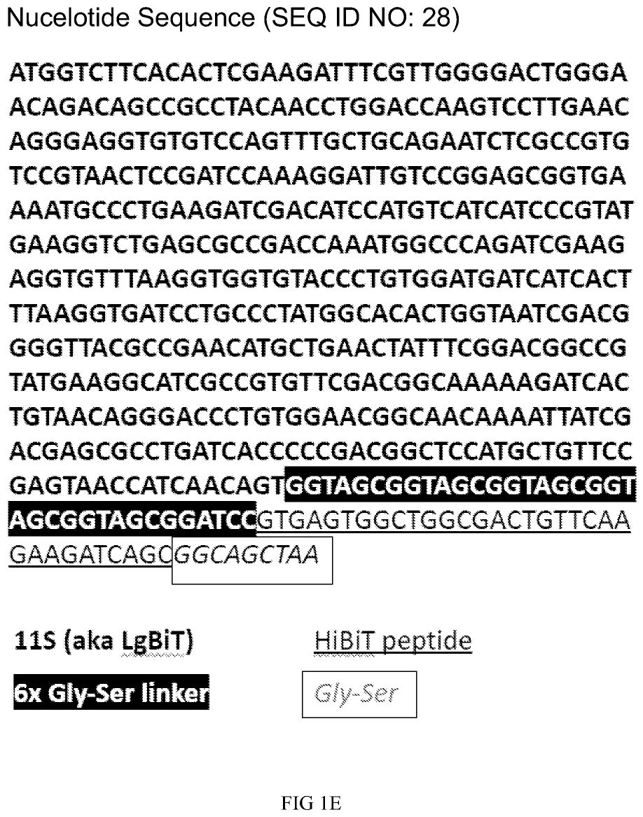

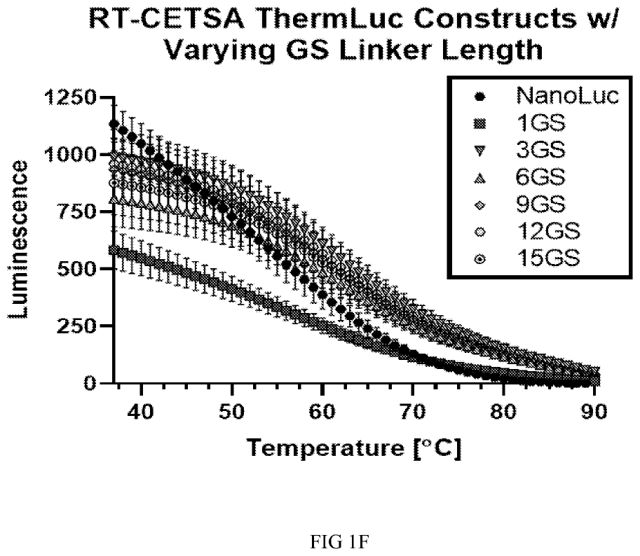

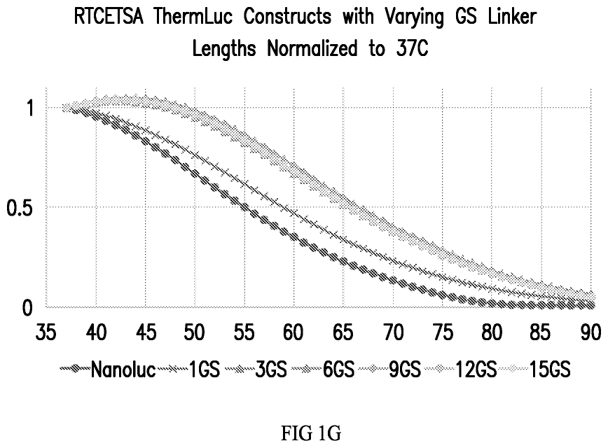

The LgBiT (11s) fragment of NanoLuc was attached to a peptide comprising the HiBit fragment of NanoLuc with a GlySer linker. This resulted in an increased melting point relative to the LgBiT fragment by itself and 156+Native peptide. (FIG. 1A.) A graphical depiction of such a reporter region is provided as FIG. 1B.

To develop the reporter molecule, the melting profile of 11s-86b fusion proteins with varying gly-ser linker lengths were obtained in cells and compared with NanoLuc. (FIGS. 1C, 1F and 1G.) When the size of the linker was increased to 3 GlySer repeats or higher between the two fragments, the resultant fusion protein displayed minimal melting over the temperature ramp range commonly used in CETSA experiments. The reporter molecule comprising the LgBiT and HiBiT-GlySer joined by a 6X GlySer linker is referred to as “ThermLuc” and the full sequences are provided as SEQ ID NO: 23 (peptide), and a nucleotide sequence encoding ThermLuc is provided as SEQ ID NO: 28. (See FIGS. 1D and 1E, respectively.)

Importantly, although ThermLuc displays a marked decrease in the luminescent signal compared to native NanoLuc (see FIG. 1C), the signal is still strong enough to enable quantification with commonly used lab Charge-coupled Devices (CCDs).

### Example 2

This example describes the development of a device suitable for conducting RT-CETSA experiments.

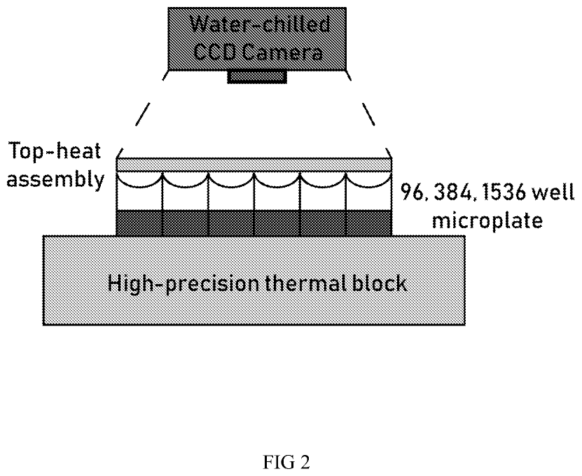

As no RT-PCR machine on the market is designed for luminescence capture, existing devices are not suitable for carrying out RT-CETSA experiments. RT-CETSA requires a high-precision and high-speed PCR thermal block capable of handling several plate formats (e.g. 96 well, 384 well, etc.), and a sensitive CCD or CMOS camera able to capture luminescence. The configuration is depicted visually in FIG. 2.

An RT-CETSA prototype was built out of a commercially-available high-throughput RT-PCR machine, the Roche LC480 (Product No. 05015278001). Excitation and emission filters were removed to maximize signal, and the camera was replaced with a water-cooled Hamamatsu Orca II CCD (C11090-22B) capable of sensitive luminescence capture. Additionally, Software tools were created to capture luminescence data from 384 well plates and perform analysis workflow to visualize real-time CETSA datasets.

### Example 3

This example describes the RT-CETSA assay and presents results obtained thereby.

In an RT-CETSA experiment, cells are transfected with a plasmid vector encoding a target protein of interest coupled with the ThermLuc reporter molecule. The target protein of interest is coupled to ThermLuc with a first linker peptide sequence GSGGGGS (SEQ ID NO: 1). The target-ThermLuc construct is then expressed in the cells.

The transfected cells are loaded onto a plate (e.g., a 96 well plate or a 384 well plate). Furimazine is then added to the samples, and the plate is exposed to a heat ramp via the high precision, high speed PCR thermal block. The luminescent signal of the intact target protein construct is captured in real time by the CCD camera. When a given temperature is reached, the target protein of interest will unfold and aggregate, and the luminescence will fade. The temperature at which 50% of the protein has aggregated is referred to as T_{agg}. Accordingly, the assay allows for full aggregation profiles of multiple proteins to be captured in parallel. Further, as heat induced aggregation can be altered by a small molecule binding to the target protein, ligand induced thermal shifts can also be observed.

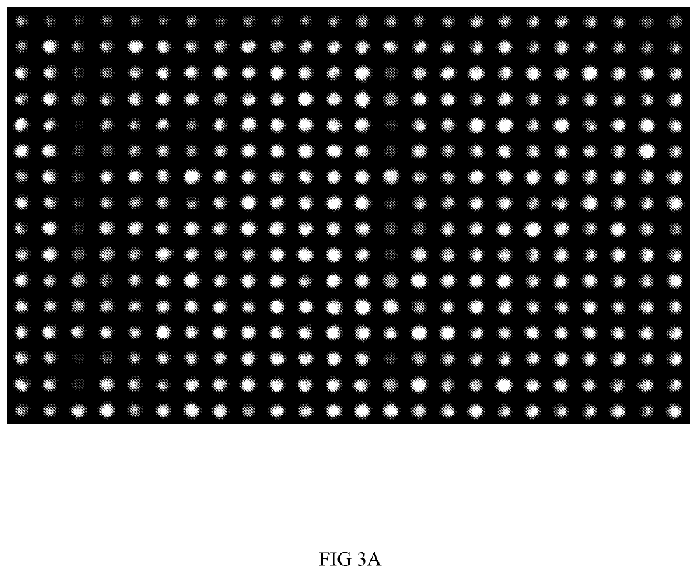

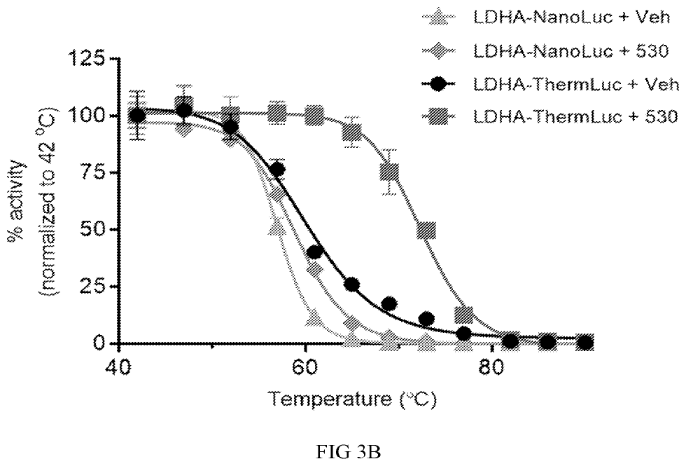

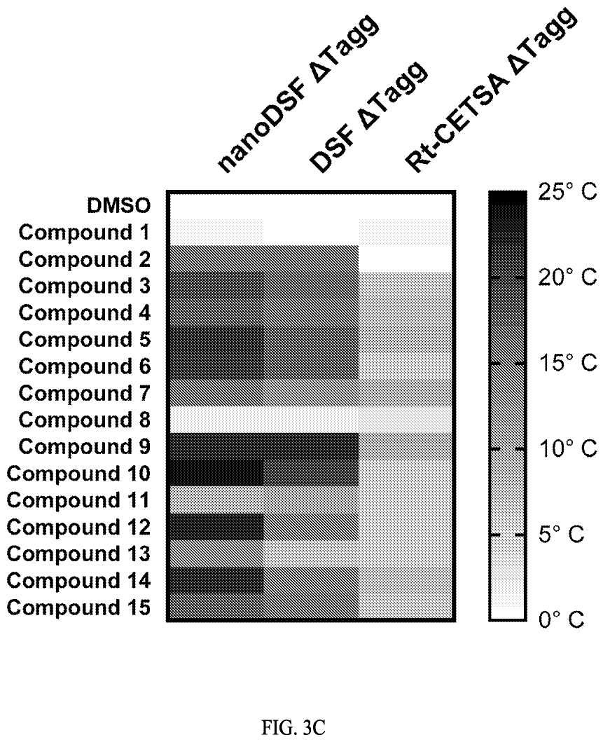

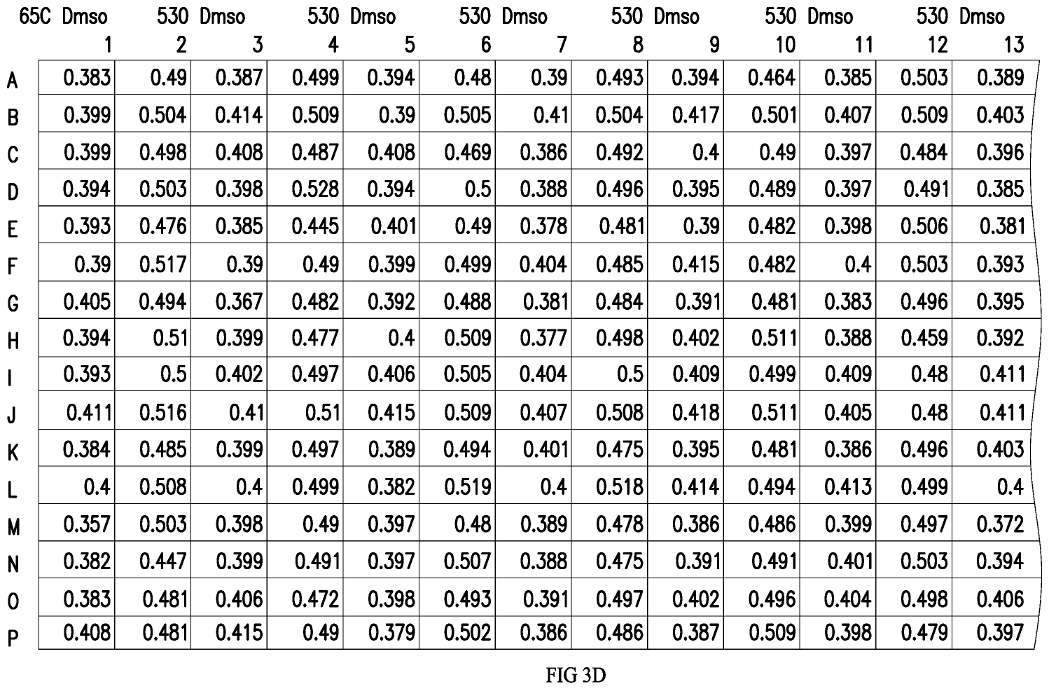

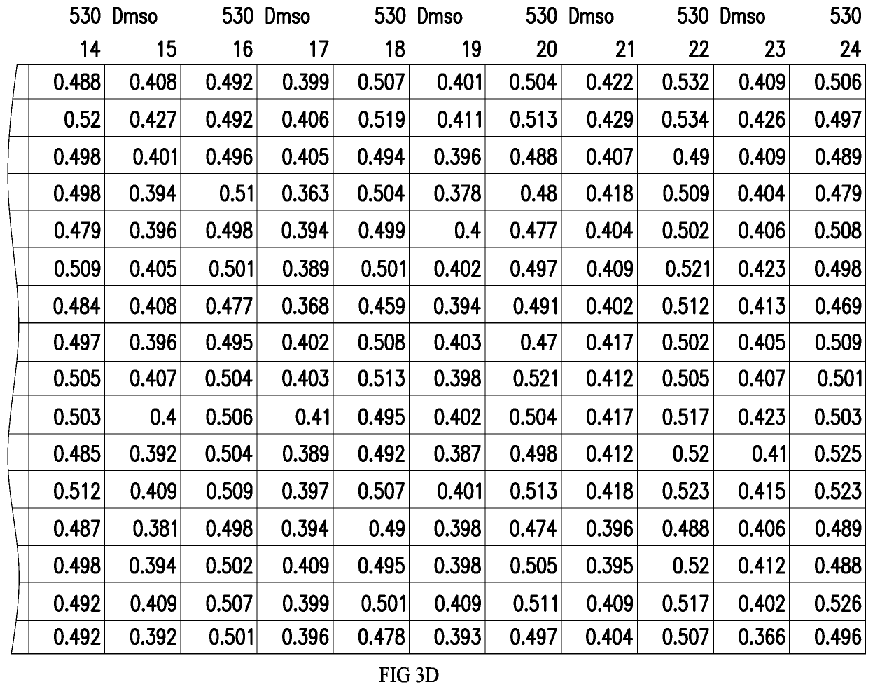

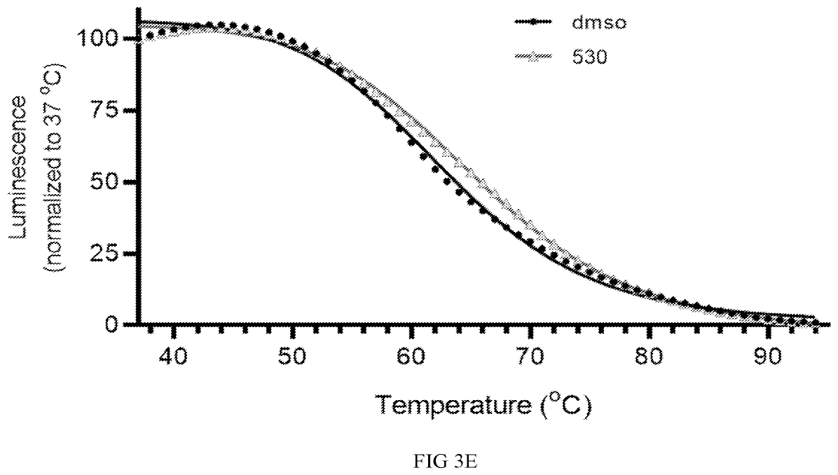

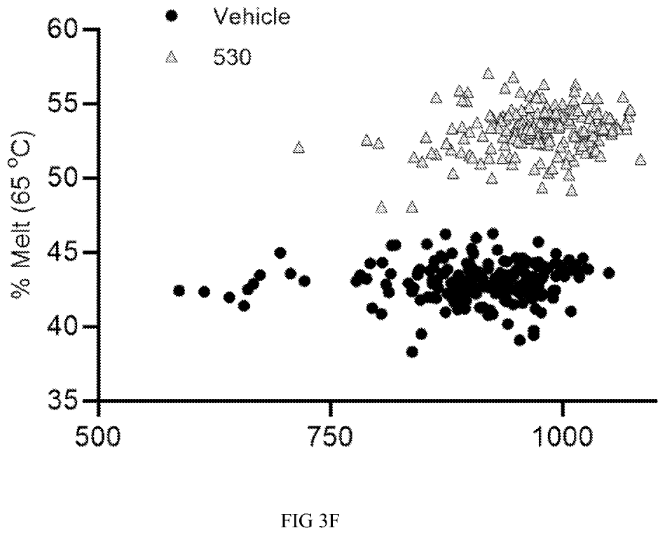

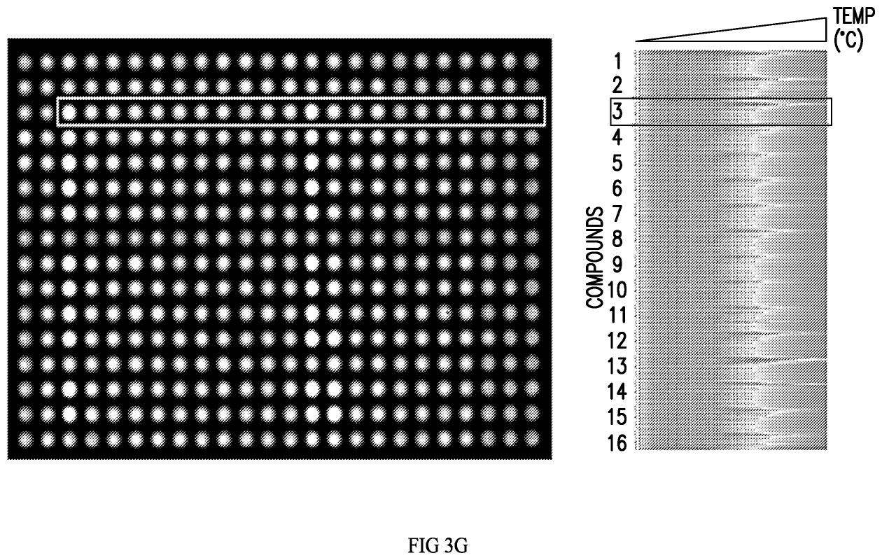

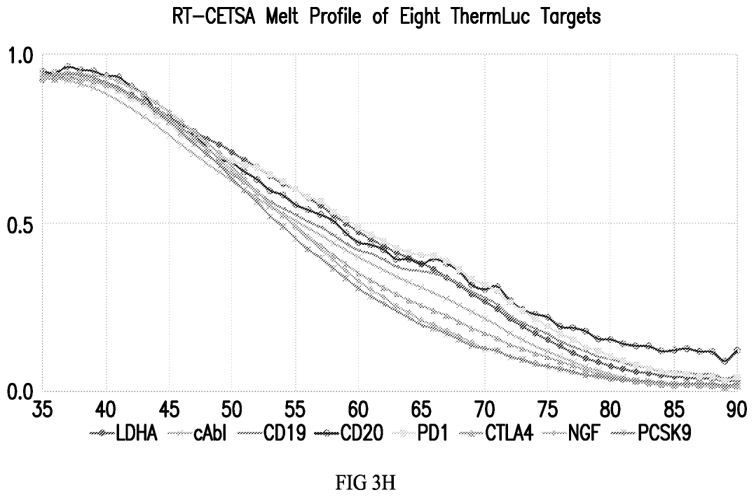

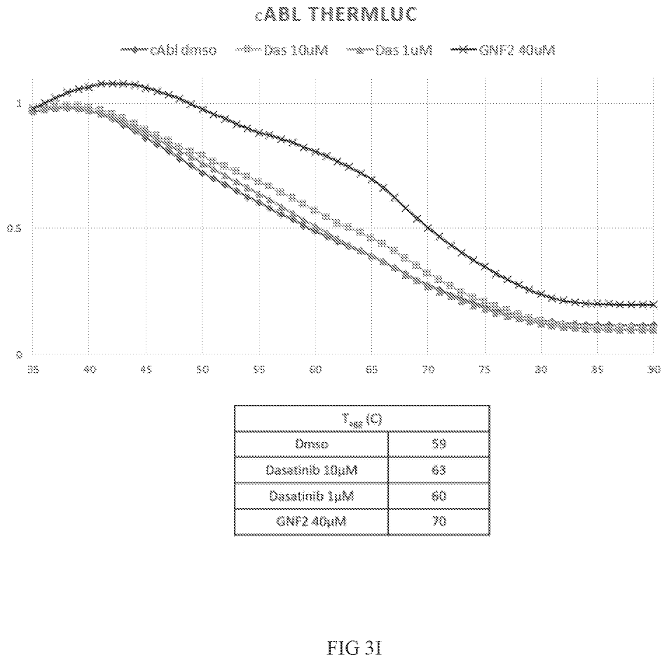

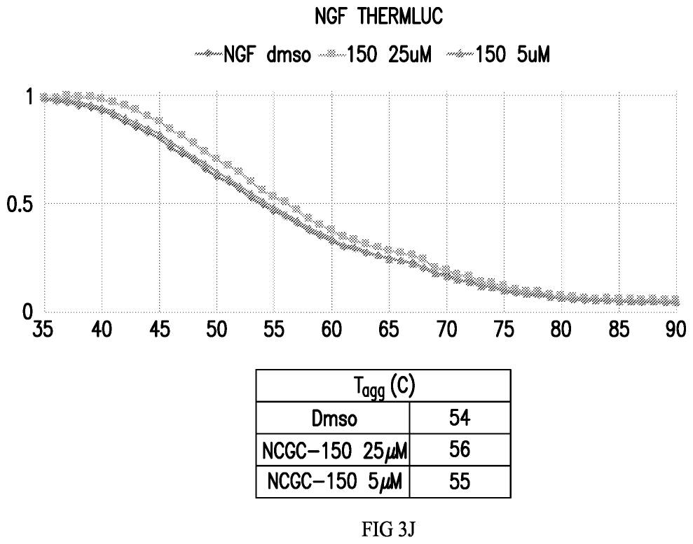

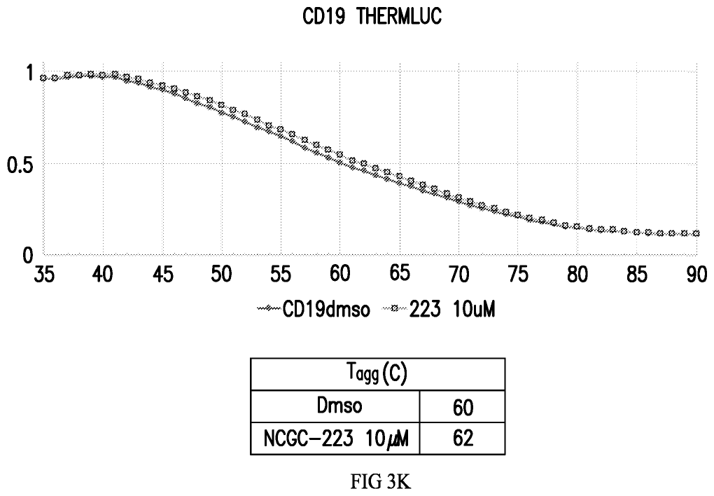

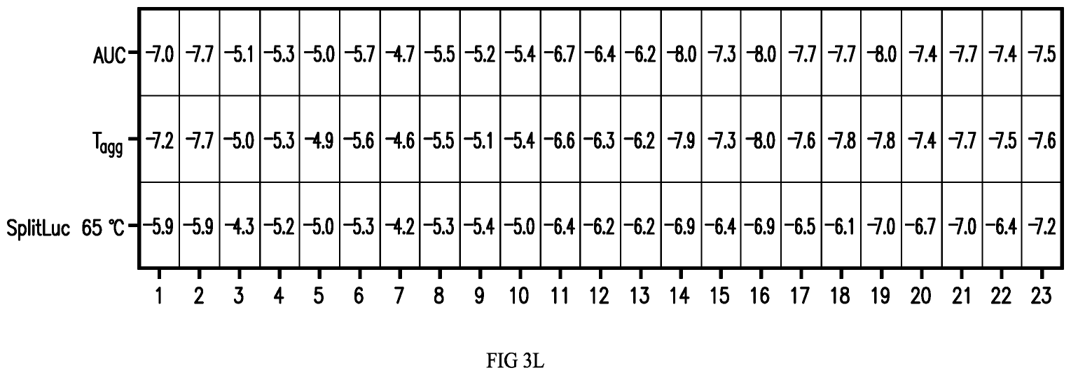

In an example of such an assay, HEK293T cells in a plate were transfected with LDHA-thermLuc plasmid and then treated with known LDHA binders and non-binders. A still image depicting the plate containing the LDHA-thermLuc transfected HEK293T cells during the assay is provided as FIG. 3A. This still image is representative of a single time point in the continuous, real-time, visualization of luminescence provided by RT-CETSA. Over the course of a RT-CETSA experiment, the observed luminescence for each well gradually decreases to background luminescence values.

Results of an experiment utilizing HEK293T cells wherein the target protein of interest is LDHA is provided as FIG. 3B. In this assay, LDHA was fused with either nanoLuciferase or ThermLuc protein and was transfected into HEK293T cells. The cells were then exposed to different temperatures. The stabilization of LDHA (T_{agg} ˜ 60° C.) with a known LDHA inhibitor is masked when using NanoLuc as a reporter, because NanoLuc is driving the aggregation of the fusion complex. Specifically, the shift in melt temperature after treatment with known binder 530 is masked (ΔTm=2.0° C.) by the lower T_{agg} of nanoLuciferase, but becomes apparent with the ThermLuc protein (ΔTm=12.5° C.). Accordingly, this assay shows that the ThermLuc protein was not the driver for aggregation of the target of interest.

RTCETSA can produce dose-response curves for small molecules against the target(s) of interest. T_{agg} values from the LDHA RT-CETSA experiment are derived from the luminescent signal and plotted against compound concentration. Multiple dose-responses are detected, in good agreement with prior art on these compounds against LDHA.

The RT-CETSA method detected binders with good correlation with other biophysical methods. The ΔT_{agg} values for the LDHA experiment produced using RT-CETSA are similar to the Tm values using differential scanning fluorimetry methods, which quantify protein melting either by detecting intrinsic amino acid fluorescence (nanoDSF) or a reporter dye (DSF). Some compounds may show binding in the DSF assay with purified protein, but not CETSA, because they do not bind the target in cells, for instance because they lack membrane permeability. These results are depicted in FIG. 3C.

Depictions of additional data obtained demonstrating the RT-CETSA assay are provided as FIGS. 3D-3K, and are described briefly, herein. FIGS. 3D-3F depict thermal shifts across a plate. FIG. 3G presents a visual depiction of data indicating that LDHA inhibitors show a dose dependent shift. FIG. 3H depicts the RT-CETSA melt profile of eight ThermLuc target proteins of interest. Finally, FIGS. 3I-3K depict target engagement in RT-CETSA using additional protein targets of interest.

As depicted in FIG. 3L, twenty-two LDHA inhibitors were tested in the RT-CETSA assay and EC50 values (log molar) were calculated using area-under-curve or T_{agg} metrics. Potency values calculated using the RT-CETSA assay (using either T_{agg} or AUC metrics) are in agreement with SplitLuc CETSA.

### Example 4

This example provides comparisons between RT-CETSA and traditional CETSA.

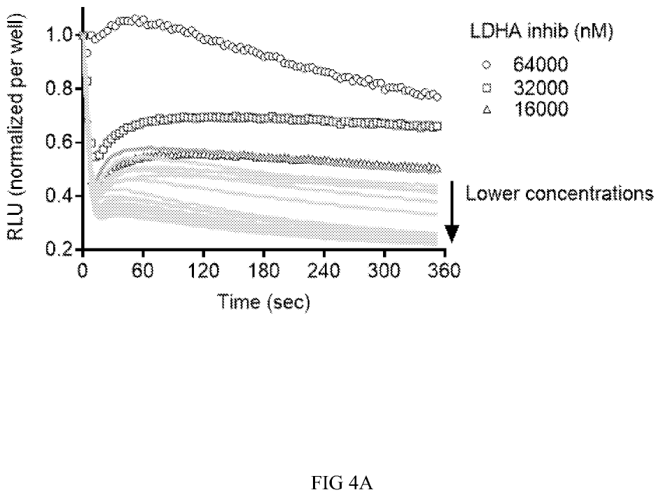

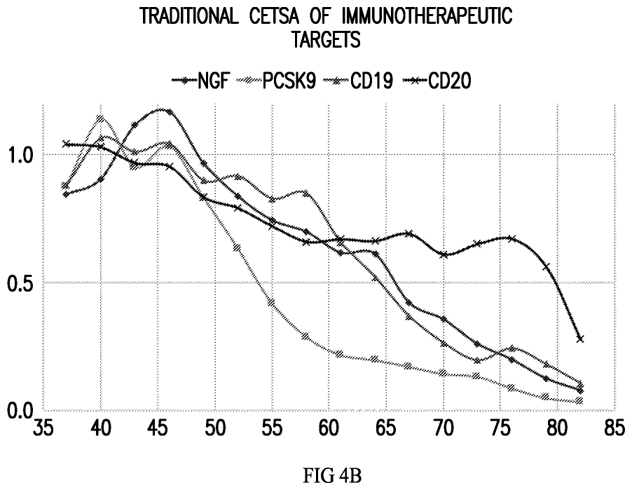

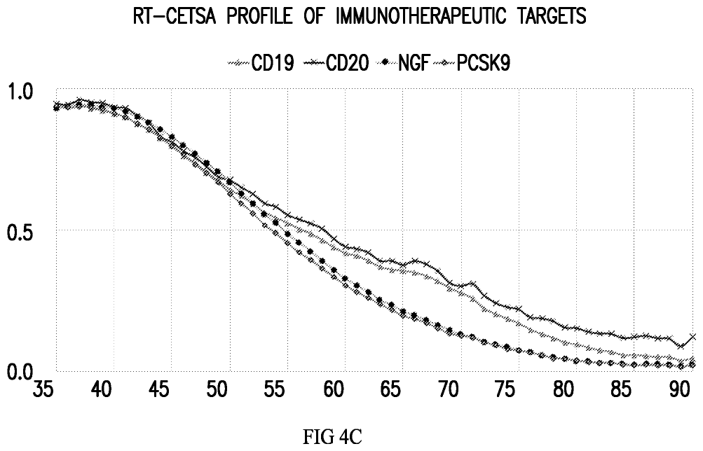

The original CETSA technique calls for application of heat for 3.5 minutes to samples. However, using the RT-CETSA method, it was demonstrated that melting of the target takes place within seconds of the application of heat. (FIG. 4A). Accordingly, RT-CETSA allows for a real-time read out of target melting that cannot be obtained with other CETSA techniques.

An additional comparison is depicted in FIGS. 4B-C. Briefly, FIGS. 4B and 4C demonstrate the profile of immunotherapeutic targets obtained with traditional CETSA as compared to RT-CETSA.

### Example 5

This example demonstrates the utility of RT-CETSA in profiling multiple target proteins.

All previous CETSA methods require optimization for each protein target, but RT-CETSA allows for multiple targets with variable melting profiles to be screened in the same experiment with less initial optimization.

RT-CETSA will allow multiple proteins (e.g., multiple members of a target class) to be profiled in the same experiment without extensive optimization. Multiple targets can be monitored in parallel even if they have different aggregation profiles. For example, data obtained from a multitarget RT-CETSA experiment will allow entire families of proteins to be profiled against a panel of compounds, i.e. a family of kinases or methyl transferases against a known activator or inhibitor for off-target or intra-family engagement.

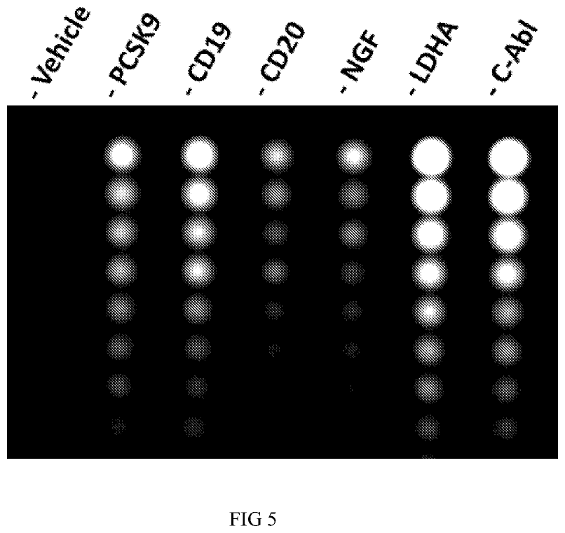

An example of a multi-target readout obtained by RT-CETSA is provided as FIG. 5.

### Example 6

This example describes the utility of a universal acceptor plasmid to improve the convenience and ease of use of RT-CETSA.

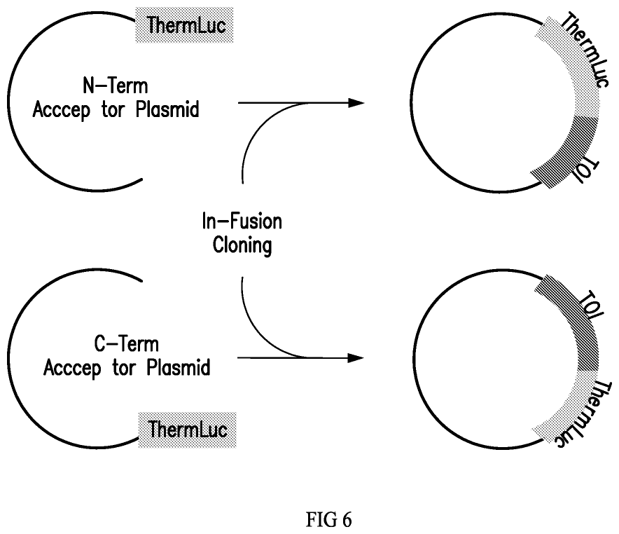

ThermLuc is inserted into a pcDNA3.1 vector with proper In-Fusion (Takara Bio) homologous sequences, containing a BamHI restriction site (encoding Gly-Ser) at the junction between ThermLuc and the target. (FIG. 6.) This will allow for researchers to easily clone in their target of interest without any unwanted, extra base pairs, to construct N-terminal or C-terminal fusion proteins. The universal acceptor plasmids expedite the process of cloning for RT-CETSA.

### Example 7

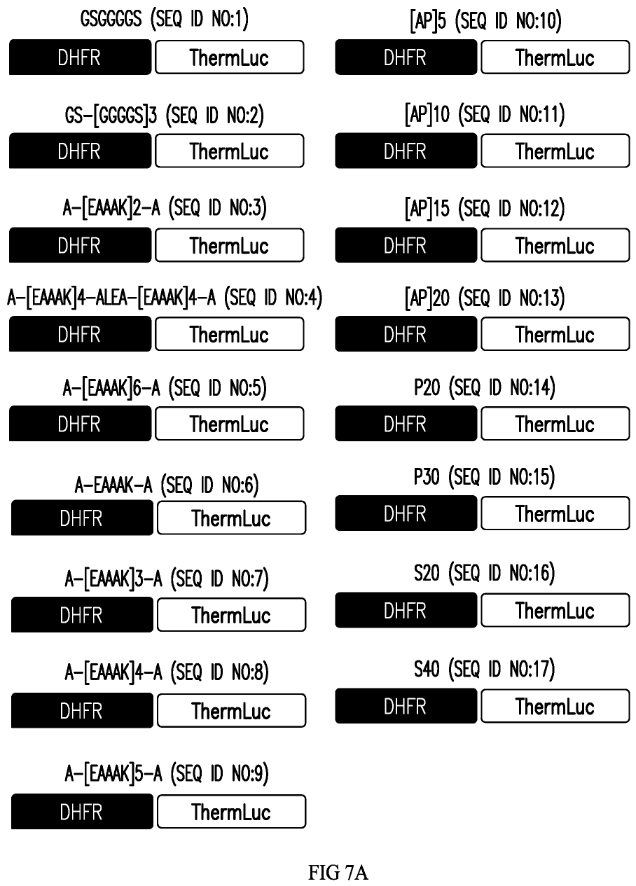

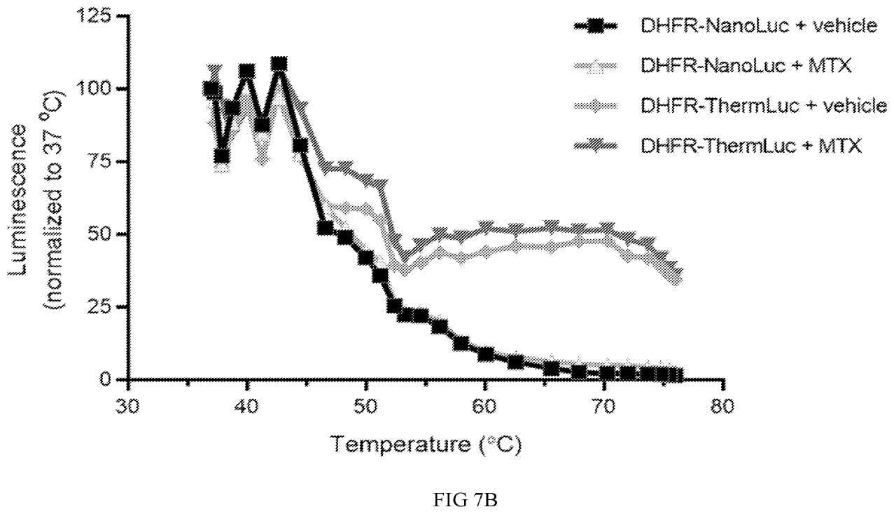

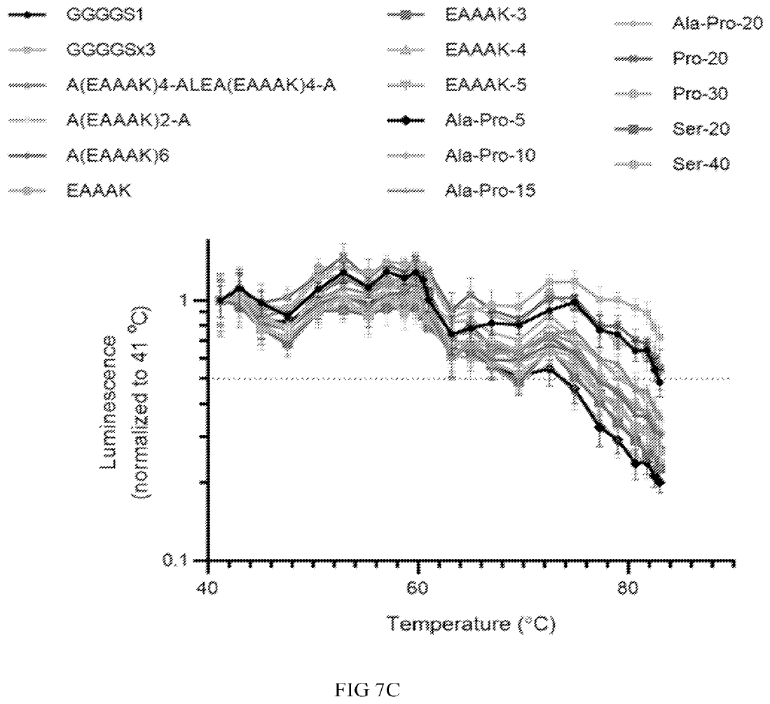

This example describes dihydrofolate reductase (DHFR)-ThermLuc reporter constructs. Various constructs were prepared with various first peptide linker region sequences between the target of interest (i.e. DHFR) and the reporter molecule as summarized in FIG. 7A.

Cellular thermal melt profiles of DHFR fusion proteins were determined. An elevated melting temperature was observed for NanoLuc and ThermLuc constructs relative to previously reported values for unlabeled or SplitLuc DHFR. However, as illustrated in FIG. 7B, only partial melting was observed in the case of the ThermLuc constructs (as indicated by the fact that higher luminescence was observed at higher temperatures relative to NanoLuc constructs). This reveals intramolecular thermal stabilization conferred by ThermLuc.

FIG. 7C is a graph depicting the thermal stability of DHFR-ThermLuc fusions when separated by various linkers/spacers (i.e. the various spacers summarized in FIG. 7A). This reveals the melting temperature of fusion proteins that show altered behavior as ThermLuc fusions with, e.g., a short GlySerGlyGlyGlyGlySer first peptide linker (SEQ ID NO: 1), can be further altered by varying first peptide linker.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the present disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, and/or exemplary language (e.g., “such as”), does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A protein construct comprising a target protein of interest, a first peptide linker, and a reporter region, wherein the reporter region comprises, from N-Terminus to C-Terminus, an LgBiT fragment, a second peptide linker, and an HiBiT fragment.

2. The protein construct of claim 1, wherein the protein construct comprises from N-terminus to C-terminus the target protein of interest, the first peptide linker, and the reporter region.

3. The protein construct of claim 1, wherein the protein construct comprises from N-terminus to C-terminus, the reporter region, the first peptide linker, and the target protein of interest.

4. The protein construct of claim 1, wherein the target protein of interest is LDHA, DHFR, cAbl, CD19, CD20, PD1, CTLA 4, NGF or PCSK9.

5. The protein construct of claim 1, wherein the first peptide linker comprises a polypeptide having at least 80% identity with any one of SEQ ID NOS: 1-17.

6. The protein construct of claim 1, wherein the LgBiT fragment comprises a polypeptide having at least 80% identity with SEQ ID NO: 18.

7. The protein construct of claim 1, wherein the second peptide linker comprises one or more GlySer repeats.

8. The protein construct of claim 7, wherein the second peptide linker comprises SEQ ID NO: 19.

9. The protein construct of claim 1, wherein the HiBiT fragment comprises a polypeptide having at least 80% identity with SEQ ID NO: 20.

10. The protein construct of claim 9 wherein the HiBiT fragment additionally comprises one or more GlySer extensions.

11. The protein construct of claim 10 wherein the HiBiT fragment comprises a polypeptide having at least 80% identity with SEQ ID NO: 21.

12. The protein construct of claim 10 wherein the HiBiT fragment comprises a polypeptide having at least 80% identity with SEQ ID NO: 22.

13. The protein construct of claim 1, wherein the reporter region comprises a polypeptide having at least 80% identity with SEQ ID NO: 23.

14. A biological vector encoding a protein construct according to claim 1.

15. The biological vector of claim 14 wherein the vector is a recombinant expression vector comprising a promoter that drives expression of the of the protein construct in mammalian cells.

16. The biological vector of claim 14, wherein the vector is a universal acceptor plasmid.

17. The biological vector of claim 14, wherein the vector is a pcDNA3.1 vector.

18. The biological vector of claim 14 wherein the vector is a viral vector.

19. The biological vector of claim 14 wherein the biological vector comprises a nucleotide sequence having greater than 80% identity to SEQ ID NO: 24.

20. The biological vector of claim 14 wherein the biological vector comprises a nucleotide sequence having greater than 80% identity to SEQ ID NOS: 25, 26, or 27.

21. The biological vector of claim 14 wherein the biological vector comprises a nucleotide sequence having greater than 80% identity to SEQ ID NO: 26.

22. The biological vector of claim 14 wherein the biological vector comprises a nucleotide sequence having greater than 80% identity to SEQ ID NO: 28.

23. A method for testing one or more samples, wherein the one or more samples comprise living intact cells, the method comprising:
a) transfecting the cells with the biological vector of claim 14 under conditions suitable to allow the expression of the protein construct within the cells;
c) exposing the expressed protein construct to a photon generating substrate; and
d) exposing the cells to an increasing temperature gradient while detecting the change in luminescence of the sample in real time.

24. The method of claim 23, wherein the photon generating substrate is furimazine.

25. The method of claim 23, further comprising contacting the protein construct with one or more additional test molecules prior to exposing the cells to the increasing temperature gradient.

26. The method of claim 25 wherein the additional test molecule is a small molecule.

27. The method of claim 25 wherein the additional test molecule binds to the target protein of interest.

28. The method of claim 25 wherein the additional test molecule is an binder (e.g inhibitor) of the target protein of interest.

29. The method of claim 25 wherein the additional test molecule is an antibody.

30. The method of claim 23 wherein two or more samples are tested in parallel.

31. The method of claim 30, wherein the two or more samples comprise different protein constructs.

32. The method of claim 30, wherein the two or more samples comprise different target proteins.

33. The method of claim 30 wherein the protein constructs of each of the two or more samples is contacted with one or more additional test molecules prior to exposing the cells to the increasing temperature gradient.

34. An analytical device; wherein the analytical device is capable of simultaneously heating and collecting real time luminescence data for multiple samples; the device comprising:
(a) a thermal cycler block adapted to receive a multi-well plate comprising the multiple samples;
(c) a detection device capable of detecting luminesce; and
(b) a thermal top-heat assembly adapted to maintain even heating across the top of the multi-well plate and to allow a luminescent signal to pass through to the detection device;
wherein the detection device is positioned such that it can detect changing luminescence in the multiple samples in real time over a range of temperature.

35. The analytical device of claim 34 wherein the detection device is a CCD sensor or a CMOS sensor.

36. The analytical device of claim 34, wherein the multi well plate is a 96-well plate, a 384-well plate or a 1,536 well plate.
